(19) United States
(12) Patent Application Publication
(10) Pub. No.: US 2022/0401597 A1
(43) Pub. Date: Dec. 22, 2022

(54) ULTRAVIOLET STERILIZATION DEVICE AND METHOD FOR CONTROLLING SAME

(71) Applicants: HYUNDAI MOTOR COMPANY, Seoul (KR); KIA CORPORATION, Seoul (KR)

(72) Inventor: Jae Seung Lee, Hwaseong-si (KR)

(73) Assignees: HYUNDAI MOTOR COMPANY, Seoul (KR); KIA CORPORATION, Seoul (KR)

(21) Appl. No.: 17/547,952

(22) Filed: Dec. 10, 2021

(30) Foreign Application Priority Data
Jun. 21, 2021 (KR) 10-2021-0079731

(51) Int. Cl. A61L 2/10; A61L 2/24; A61L 2/26

(52) U.S. Cl. CPC A61L 2/10; A61L 2/24; A61L 2/26; A61L 2202/11; A61L 2202/14; A61L 2202/16; A61L 2202/25

(57) ABSTRACT

The present disclosure relates to an ultraviolet (UV) sterilization device, and more specifically, to a UV sterilization device whose radiation mode and position may be changed. The UV sterilization device includes: a sterilization unit configured to be movable up to a specific position and including a UV emission unit; a driving unit configured to move the sterilization unit; and a lens assembly including at least two different types of lenses and provided on a front surface of the UV emission unit.

### CROSS-REFERENCE TO RELATED APPLICATION

This application claims under 35 U.S.C. § 119(a) the benefit of priority to Korean Patent Application No. 10-2021-0079731 filed on Jun. 21, 2021, the entire contents of which are incorporated herein by reference.

### BACKGROUND

### (a) Technical Field

The present disclosure relates to an ultraviolet (UV) sterilization device, and more specifically, to a UV sterilization device whose radiation mode and position may be changed.

### (b) Background Art

Ultraviolet (UV) light is used for a sterilization apparatus because it can promote chemical reactions, oxidize organic matter, and eradicate microorganisms. Recently, in particular, interest in hygiene and sterilization has increased more than ever before due to the coronavirus pandemic.

UV light includes UV-C light having a wavelength in the range of 200 to 280 nanometers (nm), UV-B light having a wavelength in the range of 280 to 315 nm, and UV-A light having a wavelength in the range of 315 to 400 nm. It is known that, when UV-C light, which has a sterilization function, is radiated onto deoxyribonucleic acid (DNA), it destroys the DNA and suppresses the regeneration and replication thereof, thus eradicating viruses. Because it has recently been reported that UV-C light is effective against coronavirus, UV-C light-emitting diode (LED) products for eradicating the coronavirus have been developed.

Due to this trend, attempts are being made to apply a UV sterilization apparatus to a vehicle in order to sterilize the passenger compartment of a vehicle, which is an enclosed space.

The above information disclosed in this Background section is only for enhancement of understanding of the background of the disclosure, and therefore it may contain information that does not form the related art that is already known in this country to a person of ordinary skill in the art.

### SUMMARY OF THE DISCLOSURE

The present disclosure has been made in an effort to solve the above problem associated with the related art.

An object of the present disclosure is to provide an ultraviolet (UV) sterilization device with improved sterilization performance and reliability.

Another object of the present disclosure is to provide a UV sterilization device capable of performing various radiation modes.

The objects of the present disclosure are not limited to the aforementioned objects, and other objects not mentioned may be clearly understood by those with ordinary skill in the art to which the present disclosure pertains (hereinafter referred to as ‘those having ordinary skill in the art’) from the following description.

The features of the present disclosure for achieving the objects of the present disclosure and performing the characteristic functions of the present disclosure to be described below are as follows.

According to some embodiments of the present disclosure, a UV sterilization device includes: a sterilization unit configured to be movable to a specific position, and including a UV emission unit; a driving unit configured to move the sterilization unit; and a lens assembly including at least two different types of lenses, and provided on a front surface of the UV emission unit.

According to some embodiments of the present disclosure, a method for controlling a UV sterilization device includes: detecting a temperature in an enclosed space in which a sterilization unit is positioned; separating the sterilization unit from an enclosure positioned in the enclosed space by driving a motor configured to move the sterilization unit from the enclosure positioned in the enclosed space when the temperature exceeds a preset threshold temperature; and driving the motor to return the sterilization unit to the enclosure positioned in the enclosed space when the temperature is equal to or lower than the threshold temperature.

The present disclosure provides the UV sterilization device with the improved sterilization performance and reliability.

The present disclosure provides the UV sterilization device capable of performing various radiation modes.

The effects of the present disclosure are not limited to the aforementioned effects, and the other effects not mentioned may be clearly recognized by those having ordinary skill in the art from the following description.

It is understood that the term “automotive” or “vehicular” or other similar term as used herein is inclusive of motor automotives in general such as passenger automobiles including sports utility automotives (operation SUV), buses, trucks, various commercial automotives, watercraft including a variety of boats and ships, aircraft, and the like, and includes hybrid automotives, electric automotives, plug-in hybrid electric automotives, hydrogen-powered automotives and other alternative fuel automotives (e.g., fuels derived from resources other than petroleum). As referred to herein, a hybrid automotive is an automotive that has two or more sources of power, for example both gasoline-powered and electric-powered automotives.

The above and other features of the disclosure are discussed below.

### BRIEF DESCRIPTION OF THE DRAWINGS

The above and other features of the present disclosure are described in detail below with reference to certain examples thereof illustrated in the accompanying drawings which are given herein below by way of illustration only, and thus are not limitative of the present disclosure, and wherein:

FIGS. 1 and 2 illustrate an example in which a UV sterilization device according to an embodiment of the present disclosure is mounted.

FIG. 3A illustrates a state where the UV sterilization device according to an embodiment of the present disclosure is mounted on an overhead console for a vehicle.

FIG. 3B illustrates a state where a position of the UV sterilization device of FIG. 3A is changed.

FIG. 4 illustrates a perspective diagram of the UV sterilization device according to an embodiment of the present disclosure.

FIG. 5 illustrates a bottom perspective diagram of FIG. 4.

FIG. 6 illustrates a sterilization unit of the UV sterilization device according to an embodiment of the present disclosure.

FIG. 7 is a cross-sectional diagram taken along line B-B′ in FIG. 6.

FIG. 8 illustrates the UV sterilization device seen from X of FIG. 5.

FIG. 9A is a partially enlarged diagram of FIG. 4.

FIG. 9B is a partially enlarged rear diagram of FIG. 4.

FIG. 10 illustrates a lens assembly according to an embodiment of the present disclosure.

FIG. 11A is a cross-sectional diagram taken along line A-A′ in FIG. 5.

FIG. 11B is a cross-sectional diagram taken along line A-A′ in FIG. 5.

FIG. 12 illustrates a configuration diagram of a control system of the UV sterilization device according to an embodiment of the present disclosure.

FIG. 13 illustrates a control flowchart of the UV sterilization device according to an embodiment of the present disclosure under a high-temperature condition.

FIG. 14A illustrates the sterilization unit according to an embodiment of the present disclosure, which is in an operation standby state in the overhead console.

FIG. 14B illustrates the sterilization unit according to an embodiment of the present disclosure moving downward from the overhead console.

FIG. 15 illustrates a user request-based control flowchart of the UV sterilization device according to an embodiment of the present disclosure.

FIG. 16A illustrates a front diagram of the UV sterilization device according to an embodiment of the present disclosure if first and second wires are set as minimum lengths.

FIG. 16B illustrate a front diagram of the UV sterilization device according to an embodiment of the present disclosure if the first and second wires are adjusted to maximum lengths.

FIG. 17 illustrates a control flowchart of the lens assembly of the UV sterilization device according to an embodiment of the present disclosure.

FIG. 18A illustrates a radiation range of the sterilization unit in a state where a convex lens overlaps a UV emission unit.

FIG. 18B illustrates a radiation range of the sterilization unit if a lens is not adopted.

FIG. 18C illustrates a radiation range of the sterilization unit in a state where a concave lens overlaps the UV emission unit.

FIG. 19A illustrates the eccentricity of light made by the UV emission unit when a part of the right of the convex lens overlaps the UV emission unit.

FIG. 19B illustrates the eccentricity of light made by the UV emission unit when a part of the left of the convex lens overlaps the UV emission unit.

It should be understood that the appended drawings are not necessarily to scale, presenting a somewhat simplified representation of various features illustrative of the basic principles of the disclosure. The specific design features of the present disclosure as disclosed herein, including, for example, specific dimensions, orientations, locations, and shapes will be determined in section by the particular intended application and use environment.

In the figures, reference numbers refer to the same or equivalent sections of the present disclosure throughout the several figures of the drawing.

### DETAILED DESCRIPTION

Hereinafter, embodiments of the present disclosure are described in detail with reference to the accompanying drawings. Specific structures or functions described in the embodiments of the present disclosure are merely for illustrative purposes. Embodiments according to the concept of the present disclosure may be implemented in various forms, and it should be understood that they should not be construed as being limited to the embodiments described in the present specification, but include all of modifications, equivalents, or substitutes included in the spirit and scope of the present disclosure.

It should be understood that, although the terms “first,” “second,” and the like may be used herein to describe various elements, these elements should not be limited by these terms. These terms are only used to distinguish one element from another element. For instance, a first element discussed below could be termed a second element without departing from the teachings of the present disclosure. Similarly, the second element could also be termed the first element.

It should be understood that when an element is referred to as being “coupled” or “connected” to another element, it can be directly coupled or connected to the other element or intervening elements may be present therebetween. In contrast, it should be understood that when an element is referred to as being “directly coupled” or “directly connected” to another element, there are no intervening elements present. Other expressions that explain the relationship between elements, such as “between,” “directly between,” “adjacent to,” or “directly adjacent to,” should be construed in the same way.

Like reference numerals denote like components throughout the specification. In the meantime, the terminology used herein is for the purpose of describing particular embodiments only and is not intended to be limiting. As used herein, the singular forms “a,” “an,” and “the” are intended to include the plural forms as well, unless the context clearly indicates otherwise. It should be further understood that the terms “comprise,” “include,” “have,” and the like, when used in this specification, specify the presence of stated components, steps, operations, and/or elements, but do not preclude the presence or addition of one or more other components, steps, operations, and/or elements thereof.

An object of the present disclosure is to provide a UV sterilization apparatus for sterilizing an enclosed indoor space, particularly the passenger compartment of a vehicle.

With regard to a UV-C LED, as the distance from an emission source to a target to be sterilized increases, or depending on an emission angle, the intensity of light decreases, and the sterilization performance is degraded. Therefore, the longer the distance between the UV-C LED and the target to be sterilized, the longer the emission time.

Further, a UV-C LED is less effective and is more prone to failure in a high-temperature environment. When a UV-C LED operates in a high-temperature environment, for example, such as when a vehicle is parked under direct sunlight, the effect of the LED may be degraded, or may break down due to high temperatures.

In general, in the passenger compartment of a vehicle, large amounts of bacteria are present on a steering wheel provided in front of a driver's seat, door handles, seat belts, cup holders, a gear selector, and a center fascia. Therefore, the sterilization apparatus needs to be installed in a vehicle so that the sterilization effect is applied to various points in the passenger compartment of the vehicle.

An object of the present disclosure is to provide a UV sterilization apparatus and a method of controlling the same capable of preventing degradation of sterilization performance due to an increase in the distance to a target to be sterilized, of preventing deterioration in the performance of the sterilization apparatus in a high-temperature environment and of providing the sterilization effect throughout various areas in a vehicle.

Hereinafter, the present disclosure is described in detail with reference to the accompanying drawings.

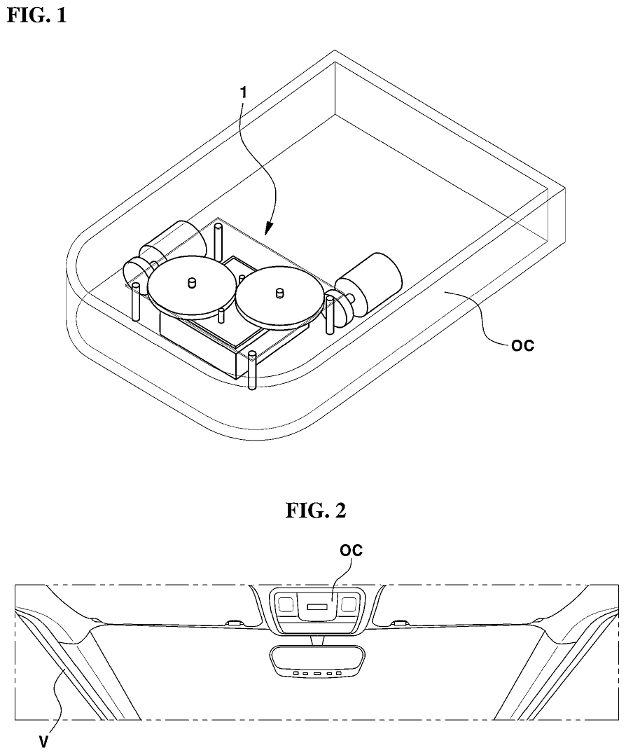

As shown in FIG. 1, a UV sterilization apparatus 1 according to the present disclosure may be used in any indoor space requiring sterilization. In particular, the UV sterilization apparatus according to the present disclosure may achieve sterilization more effectively when used in a confined indoor space, for example, the passenger compartment of a vehicle.

The UV sterilization apparatus 1 according to the present disclosure may be mounted at any position in a vehicle V. In particular, as shown in FIG. 2, the UV sterilization apparatus 1 may be mounted to an upper side in the passenger compartment of the vehicle V, for example, the roof, the head lining, or the overhead console OC of the vehicle V. Hereinafter, the case in which the UV sterilization apparatus 1 is mounted in the overhead console OC is described by way of example. However, the UV sterilization apparatus 1 is not necessarily mounted in the overhead console OC, and may be mounted at any position, so long as the apparatus 1 is capable of vertically moving.

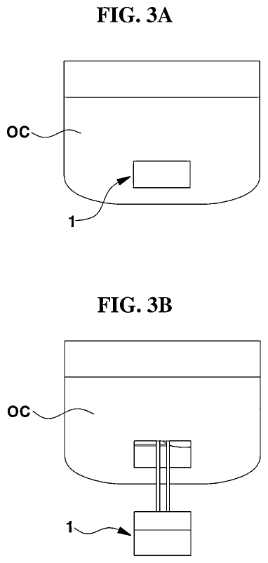

As shown in FIGS. 3A and 3B, the UV sterilization apparatus 1 according to the present disclosure is configured to be movable. The UV sterilization apparatus 1 is configured to be capable of ascending and descending in the state of being mounted in the overhead console OC. In particular, the UV sterilization apparatus 1 is formed to be movable upwards and downwards in order to satisfy temperature conditions and/or improve sterilization performance This is described below.

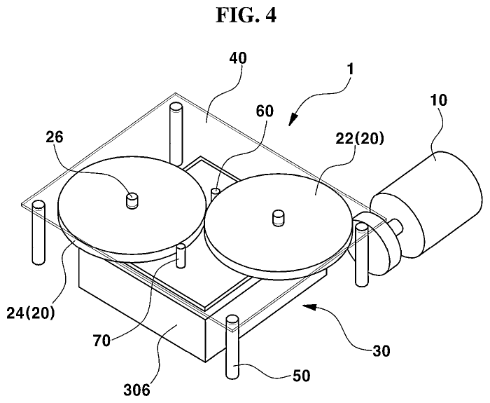

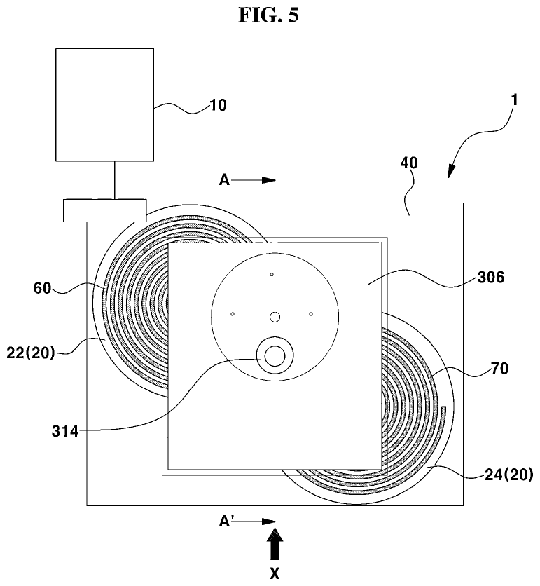

As shown in FIGS. 4 and 5, according to an embodiment of the present disclosure, the UV sterilization apparatus 1 includes a motor 10, a pulley 20, and a sterilization unit 30 to ascend and descend. The motor 10 and the pulley 20 are fixed, and the sterilization unit 30 is configured to be moved upwards and downwards by the operation of the motor 10 and the pulley 20.

The motor 10 and the pulley 20 are fixed in the overhead console OC, and the motor 10 provides driving force to the pulley 20. According to an embodiment of the present disclosure, the pulley 20 includes a first pulley 22 and a second pulley 24.

The pulley 20 may be fixed in the overhead console OC through a fixing panel 40. The fixing panel 40 is coupled to the overhead console OC through a support pin 50. Each of the first pulley 22 and the second pulley 24 is rotatably mounted to the fixing panel 40 through a pulley pin 26. In addition, the pulley 20 is configured such that, when the first pulley 22 is rotated, the second pulley 24 is also rotated together therewith. More specifically, when the motor 10 provides rotational force to the first pulley 22, the second pulley 24, which is engaged with the first pulley 22, is also rotated together therewith.

A first wire 60 is wound around the first pulley 22. When the motor 10 rotates in a first direction to rotate the first pulley 22, the first wire 60 wound around the first pulley 22 may be unwound therefrom. Similarly, a second wire 70 is wound around the second pulley 24. As the second pulley 24 is rotated in association with the first pulley 22, the second wire 70 wound around the second pulley 24 is unwound therefrom. The first wire 60 may be one of an anode wire and a cathode wire, and the second wire 70 may be the other one of the anode wire and the cathode wire.

In addition, when the motor 10 rotates in a second direction, which is opposite the first direction, to rotate the first pulley 22, the unwound first wire 60 may be wound around the first pulley 22 again. Similarly, as the second pulley 24 is rotated in association with the first pulley 22, the unwound second wire 70 may be wound around the second pulley 24 again.

The first wire 60 and the second wire 70 are connected to the sterilization unit 30 to supply power to the sterilization unit 30. The first wire 60 and the second wire 70 are respectively unwound from or wound around the first pulley 22 and the second pulley 24 by the operation of the motor 10 and the first and second pulleys 22 and 24, thereby making it possible to change the position of the sterilization unit 30 with respect to the overhead console OC.

The sterilization unit 30 includes a UV emitter 302, configured to generate a UV ray to perform sterilization. According to an embodiment of the present disclosure, the UV emitter 302 is a light-emitting diode (LED) configured to generate UV radiation, such as, for example, UV-C radiation.

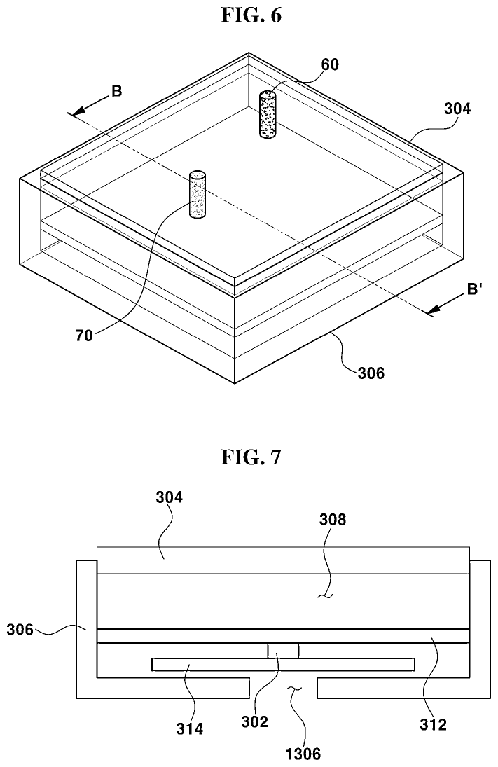

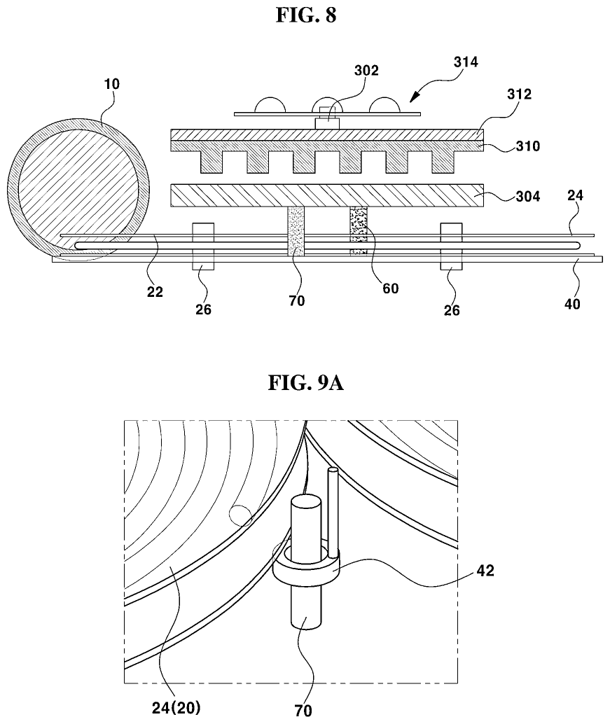

Referring to FIGS. 6-8, the sterilization unit 30 includes an inner cover 304. The inner cover 304 is disposed to face the pulley 20. In particular, the first wire 60 and the second wire 70 may be coupled to the inner cover 304. Accordingly, the sterilization unit 30 may ascend or descend due to a change in the length of the first wire 60 and the second wire 70. The inner cover 304 may be made of an aluminum material, which has excellent heat dissipation performance, but an embodiment is not limited thereto.

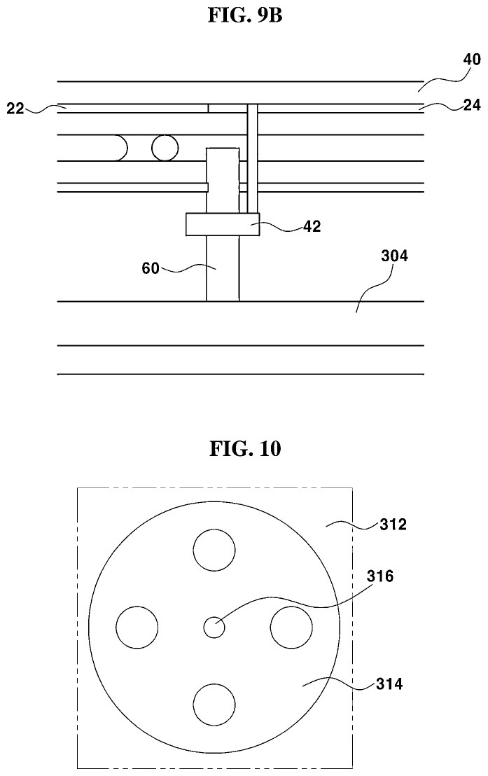

Referring to FIGS. 9A and 9B, according to an embodiment of the present disclosure, at least two wire-fixing rings 42 may be mounted to the fixing panel 40. One of the wire-fixing rings 42 serves to guide the first wire 60 extending from the first pulley 22. Similarly, another one of the wire-fixing ring 42 guides the second wire 70 extending from the second pulley 24.

Referring again to FIG. 7, the inner cover 304 is coupled to an outer cover 306, and a space 308 is formed between the inner cover 304 and the outer cover 306. The outer cover 306 accommodates the components of the sterilization unit 30, and an opening 1306 is formed in the bottom or in the center of the lower surface of the outer cover 306. The light generated by the UV emitter 302, which is accommodated in the outer cover 306, is emitted to the outside of the sterilization unit 30 through the opening 1306.

A printed circuit board 312 including a heat sink 310 for heat dissipation is disposed in the space 308. The printed circuit board 312 is configured to receive instructions from a controller 80, such as a vehicle controller, and to control the operation of the UV emitter 302 disposed under the printed circuit board 312.

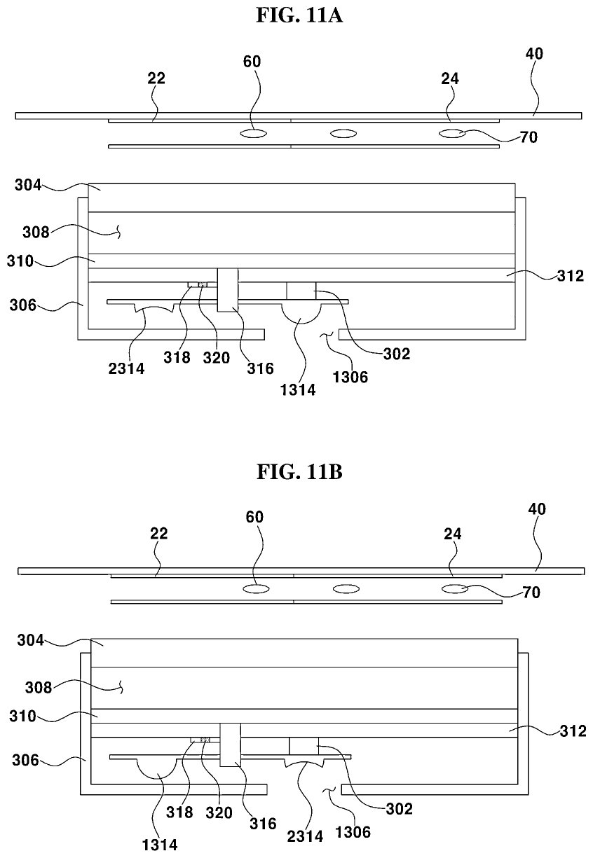

A lens assembly 314 is disposed under the UV emitter 302 in the space 308. FIG. 10 illustrates the lens assembly 314 according to an embodiment of the present disclosure. The lens assembly 314 is rotatably provided. Specifically, the lens assembly 314 is rotatably coupled to the printed circuit board 312.

To this end, according to an implementation example of the present disclosure, as illustrated in FIGS. 11A and 11B, the sterilization unit 30 includes a lens pin 316 that connects the printed circuit board 312 to the lens assembly 314. One side of the lens pin 316 is coupled to the printed circuit board 312, and the other side of the lens pin 316 is coupled to the lens assembly 314. The lens pin 316 may be configured by an external power source to be rotatable with respect to the printed circuit board 312 to rotate the lens assembly 314. As the external power source, for example, a coin motor 318 may be used. The coin motor 318 is mounted on the printed circuit board 312 and provides a rotating force to the lens pin 316. According to an implementation example of the present disclosure, the coin motor 318 may be mounted on the printed circuit board 312 through a coin motor pin 320.

As illustrated in FIG. 10, the lens assembly 314 may be formed in a plate form and includes at least two different types of lenses. The lens may include a convex lens and a concave lens, or include various patterns of lenses, such as a wavy pattern.

Each lens is set at a position where each lens may overlap the UV emitter 302 while the lens assembly 314 rotates. In other words, the present disclosure may apply various lenses to adjust the sterilization range.

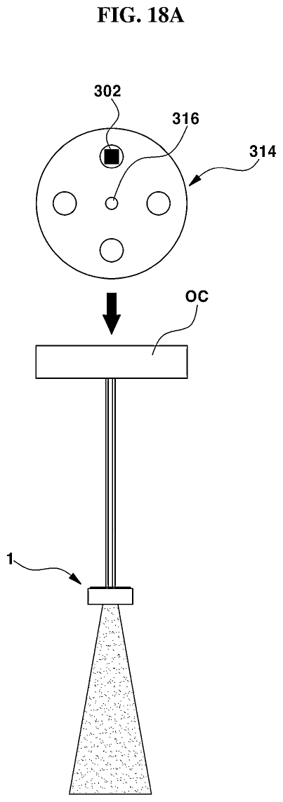

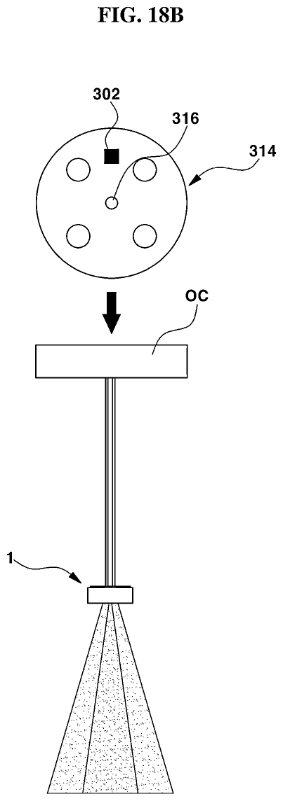

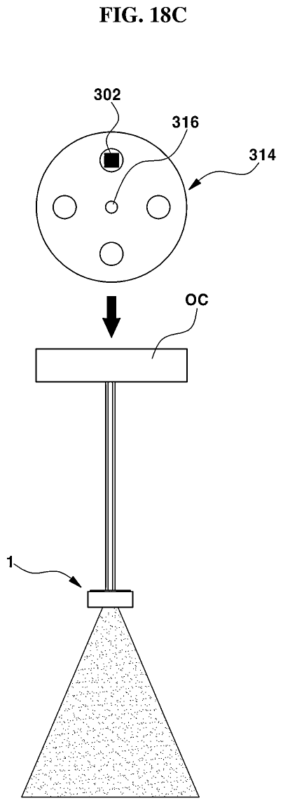

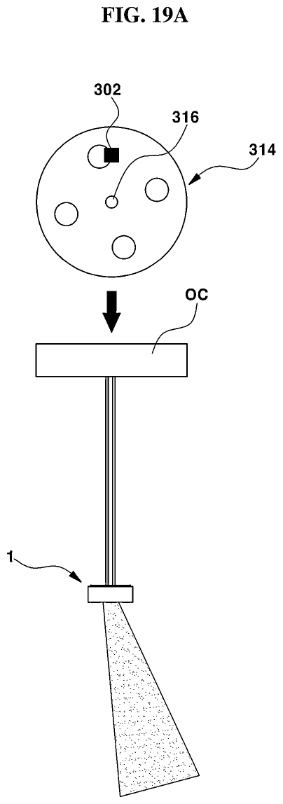

For example, if the lens assembly 314 is rotated such that a convex lens 1314 overlaps the UV emitter 302 (see FIG. 11A), the convex lens 1314 may make UV radiation energy emitted by the UV emitter 302 uniform and provide stronger light in a narrow radiation range. When the convex lens 1314 is applied, it is possible to maximize the energy of the UV emitter 302 through intensive radiation, and to intensively radiate high energy, thereby increasing remote sterilization performance (see FIG. 18A).

Further, for example, if the lens assembly 314 is rotated such that a concave lens 2314 overlaps the UV emitter 302 (see FIG. 11B), the uniform, weak UV radiation energy may be applied to the sterilization target widely. In other words, through the concave lens 2314, it is possible to radiate constant energy to a wide region, thereby increasing an application range of the UV emitter 302, and to radiate low energy constantly and widely, thereby increasing sterilization performance for a wide range (see FIG. 18C).

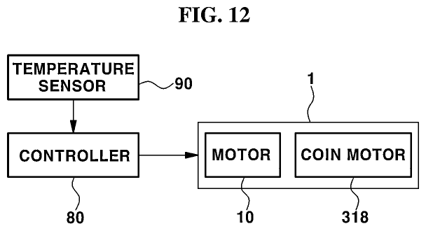

As shown in FIG. 12, the UV sterilization apparatus 1 according to the present disclosure further includes a controller 80. The controller 80 is configured to move the position of the sterilization unit 30 and to select a lens of the lens assembly 314 in response to the input conditions. The input conditions include the temperature in the passenger compartment of a vehicle and/or a request from an occupant of a vehicle. The controller 80 is configured to drive the motor 10 in order to move the position of the sterilization unit 30 and to drive the coin motor 318. In addition, the controller 80 is configured to communicate with a temperature sensor 90, which is configured to measure the temperature in the passenger compartment of a vehicle. Hereinafter, the operation of the controller 80 is described.

The UV sterilization apparatus 1 according to the present disclosure is automatically operated as follows. The UV sterilization apparatus 1 according to the present disclosure may be configured to automatically move in order to prevent deterioration in the performance thereof in a high-temperature environment, i.e., when the temperature in the passenger compartment of a vehicle exceeds a predetermined level.

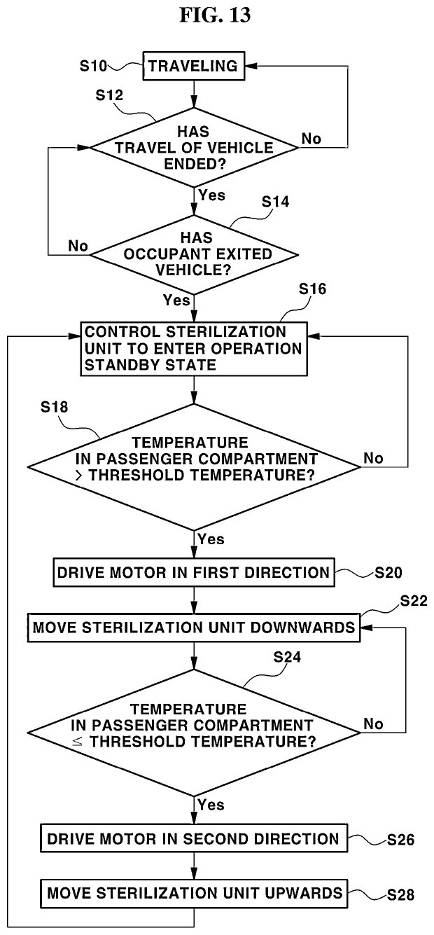

Referring to FIG. 13, the controller 80 determines whether the travel of the vehicle has ended (S10 and S12). For example, the controller 80 may sense whether the travel of the vehicle has ended through communication with the vehicle controller. At the end of the travel of the vehicle, the sterilization unit 30 is inserted into the overhead console OC. When the occupant gets out of the vehicle, the sterilization unit 30 enters an operation standby state.

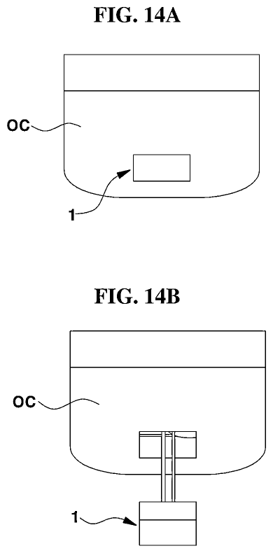

Upon determining that the travel of the vehicle has ended, the controller 80 determines whether there is an occupant in the vehicle (S14). Upon determining that there is no occupant in the vehicle, as shown in FIG. 14A, the sterilization unit 30 enters the operation standby state (S16), at which point the sterilization unit 30 is located in the overhead console OC.

When the sterilization unit 30 is in the operation standby state, the controller 80 receives information about the temperature in the passenger compartment of the vehicle in real time from the temperature sensor 90. For example, when the outdoor temperature is high and the vehicle is parked outdoors, the temperature in the indoor space of the vehicle increases, and the temperature of the sterilization unit 30, which is located in the enclosed space, also increases. Accordingly, upon determining that the temperature in the passenger compartment of the vehicle exceeds a predetermined threshold temperature, for example, 30 degrees Celsius, based on the temperature information received from the temperature sensor 90 (S18), the controller 80 controls the motor 10 to be driven. The controller 80 drives the motor 10 in the first direction so that the sterilization unit 30 descends (S20). The first pulley 22 and the second pulley 24 are rotated by the operation of the motor 10 so that the sterilization unit 30 descends (S22). The sterilization unit 30 descends and is located outside the overhead console OC, as shown in FIG. 14B.

In the state in which the sterilization unit 30 is moved downwards, the controller 80 continuously receives the information about the temperature in the passenger compartment of the vehicle from the temperature sensor 90 and determines whether the temperature in the passenger compartment of the vehicle is lower than or equal to the threshold temperature (S24). Upon determining that the temperature in the passenger compartment of the vehicle is lower than or equal to 30 degrees Celsius, which is the threshold temperature, the controller 80 drives the motor 10 again so that the sterilization unit 30 ascends to the original position thereof. In other words, the controller 80 drives the motor 10 in the second direction, which is opposite the first direction (S26), whereby the sterilization unit 30 ascends to the position shown in FIG. 14A (S28). The present disclosure functions to prevent the sterilization unit 30 from getting hot in the confined space in the overhead console OC in a high-temperature environment and to remove heat from the sterilization unit 30 by moving the same downwards. Accordingly, it is possible to prevent deterioration in the sterilization performance of the sterilization unit 30 due to high temperatures and to improve the durability thereof.

According to an embodiment of the present disclosure, the UV sterilization apparatus 1 may be moved when performing sterilization in response to a request from an occupant. The controller 80 is configured to enter the sterilization mode in response to the request from the occupant.

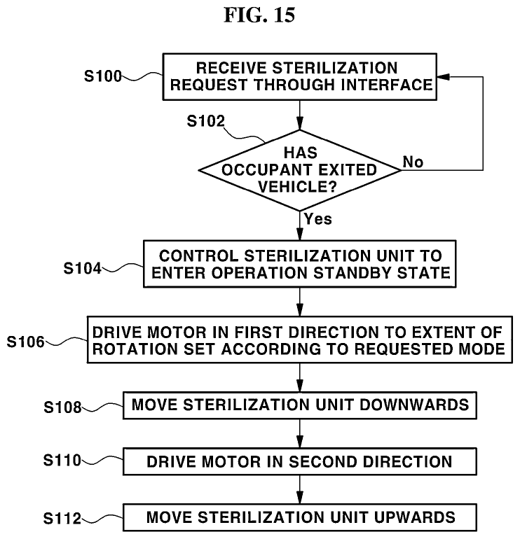

As shown in FIG. 15, the request from the occupant is transmitted to the controller 80 through an interface, such as an audio-video-navigation (AVN) system of the vehicle or a smart device configured to communicate with the vehicle (S100). The request from the occupant may request any of several modes. For example, the requested sterilization mode may include at least a broad-range sterilization mode, an intensive sterilization mode, and an intermediate sterilization mode. The controller 80 may drive the motor 10 according to each mode to adjust the descending lengths of the first wire 60 and the second wire 70.

Upon receiving the sterilization request from an occupant, the controller 80 determines whether there is an occupant in the vehicle (S102). Upon determining that there is no occupant in the vehicle, the sterilization unit 30 enters the operation standby state in which the sterilization unit 30 can move (S104).

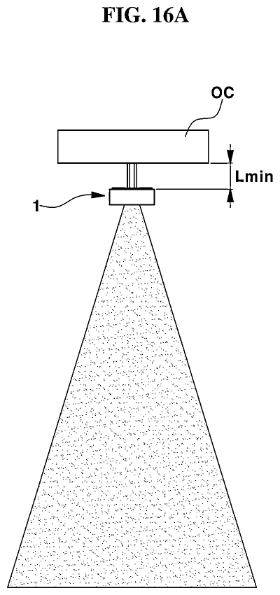

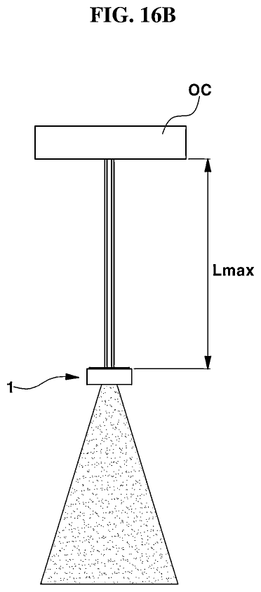

According to each mode, the descending distance of the sterilization unit 30 may be set by adjusting the lengths that the first wire 60 and the second wire 70 are withdrawn out of the overhead console OC. As shown in FIGS. 16A and 16B, according to the requested sterilization mode, the controller 80 controls the withdrawal lengths of the first and second wires 60 and 70 between the minimum lengths Lmin (FIG. 16A) and the maximum lengths Lmax (FIG. 16B) of the first and second wires 60 and 70, thereby enabling sterilization suitable for the requested sterilization mode. In other words, the controller 80 may drive the motor 10 in the first direction to the extent of rotation set according to the requested mode (S106), whereby the sterilization unit 30 descends a predetermined distance (S108).

For example, when the occupant requests broad-range sterilization, the sterilization unit 30 descends a relatively short distance from the overhead console OC (FIG. 16A). On the other hand, when the occupant requests intensive sterilization, the sterilization unit 30 descends the predetermined maximum descending distance from the overhead console OC (FIG. 16B). In addition, when the occupant requests intermediate sterilization, the sterilization unit 30 descends a distance suitable therefor. The sterilization performance of the sterilization apparatus depends on the distance to the target to be sterilized. According to the present disclosure, it is possible to secure excellent sterilization performance by moving the UV sterilization apparatus 1 close to the target to be sterilized. In addition, the present disclosure provides the broad-range sterilization function and the intensive sterilization function in response to the selection of a user, thereby providing diversity and convenience in sterilization.

After the sterilization unit 30 descends and performs sterilization for a predetermined time, the controller 80 drives the motor 10 in the second direction (S110). Thereby, the sterilization unit 30 ascends and is inserted into the overhead console OC to return to the original position thereof (S112).

According to some embodiments of the present disclosure, more minute and various sterilization controls may be possible by controlling the lens assembly 314.

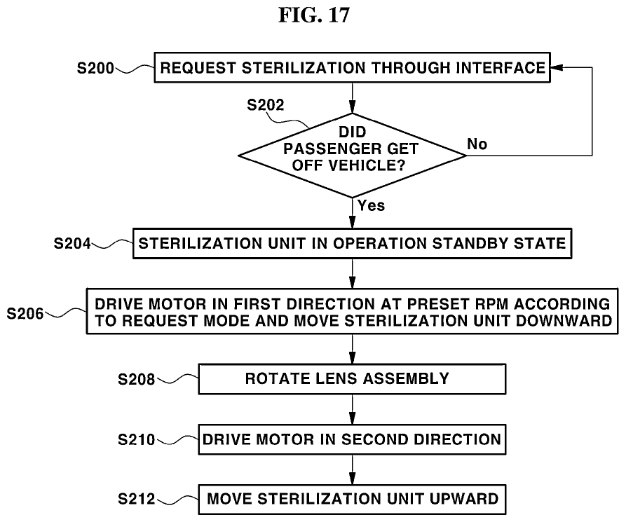

Referring to FIG. 17, in a step S200, the passenger of the vehicle selects and requests the sterilization mode of the vehicle through an interface, and the controller 80 receives the passenger's selection and request.

Next, the controller 80 confirms whether there is a passenger in the vehicle (S202). If it is determined that there is no passenger, the sterilization unit 30 is placed in the operation standby state in which the sterilization unit 30 is movable (S204).

The sterilization unit 30 moves to perform a mode matched with the passenger's request under the control of the controller 80 (S206). Further, the controller 80 drives the coin motor 318 to rotate the lens assembly 314 such that a lens suitable for the requested sterilization mode is selected (S208). For example, the convex lens 1314, the concave lens 2314, or no lens may be selected. As illustrated in FIG. 18A, if the intensive radiation mode is requested, the controller 80 rotates the lens assembly 314 such that the convex lens 1314 overlaps the UV emitter 302. As illustrated in FIG. 18B, no lens may be selected. Further, as illustrated in FIG. 18C, if the wide-range radiation mode is requested, the controller 80 rotates the lens assembly 314 such that the concave lens 2314 overlaps the UV emitter 302.

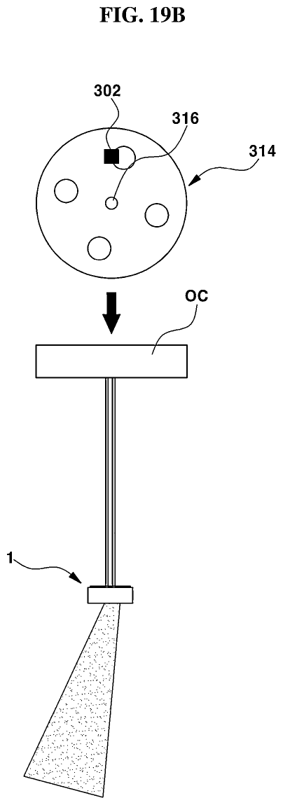

Further, in some modes, a UV radiation angle may be adjusted by overlapping only a part of the selected lens with the UV emitter 302. The controller 80 adjusts the rotating angle of the lens assembly 314, for example, allows only about right half of the convex lens 1314 to overlap the UV emitter 302 (see FIG. 19A). In this case, it is possible to expect the eccentric effect on the right. Further, the controller 80 may adjust the rotating angle of the lens assembly 314 such that only about left half of the convex lens 1314 overlaps the UV emitter 302, thereby providing the eccentric effect on the left (see FIG. 19B).

After the sterilization mode is performed according to the user's request, the controller 80 drives the motor 10 in the second direction (S210) to move the sterilization unit 30 upward to return the sterilization unit 30 to the original position (S212).

According to the present disclosure, the sterilization unit 30 may be configured to be movable such that the distance between the sterilization target and the sterilization unit is adjusted, which makes it possible to improve sterilization performance

According to the present disclosure, the sterilization unit 30 may be separated from the overhead console (OC) when the UV sterilization device 1 is placed in the high-temperature condition, which makes it possible to achieve cooling.

According to the present disclosure, the lens assembly 314 may be included such that various UV light radiation modes are provided, which makes it possible to improve sterilization performance and the user's convenience and satisfaction.

The aforementioned present disclosure is not limited to the aforementioned embodiments and the accompanying drawings, and it is apparent to those having ordinary skill in the art to which the present disclosure pertains that various substitutions, modifications, and changes may be made without departing from the technical spirit of the present disclosure.

What is claimed is:

1. An ultraviolet (UV) sterilization device comprising:
a sterilization unit configured to be movable to a specific position, and comprising a UV emission unit;
a driving unit configured to move the sterilization unit; and
a lens assembly comprising at least two different types of lenses, and provided on a front surface of the UV emission unit.

2. The UV sterilization device of claim 1,
wherein the driving unit comprises:
a motor configured to provide a rotating force; and
a pulley rotated by the motor and having a wire connected to the sterilization unit and wound therearound to be unwoundable.

3. The UV sterilization device of claim 2,
wherein the pulley comprises:
a first pulley configured to directly receive a rotating force from the motor and having a first wire wound therearound; and
a second pulley configured to rotate in engagement with the first pulley and having a second wire wound therearound.

4. The UV sterilization device of claim 2, further comprising: a wire fixing ring configured to guide the wire unwound from the pulley.

5. The UV sterilization device of claim 1,
wherein the lens assembly is rotatably mounted on the sterilization unit.

6. The UV sterilization device of claim 5,
wherein the lens assembly comprises: at least one lens of a convex lens and a concave lens

7. The UV sterilization device of claim 2,
wherein the sterilization unit comprises:
a cover connected to the wire; and
a printed circuit board accommodated in the cover, configured to control an operation of the UV emission unit, and having the UV emission unit mounted thereon.

8. The UV sterilization device of claim 7,
wherein the lens assembly is rotatably coupled to the printed circuit board.

9. The UV sterilization device of claim 8,
wherein the lens assembly receives a rotating force from a coin motor mounted on the printed circuit board.

10. The UV sterilization device of claim 7,
wherein an offset exists between the center of the lens assembly and the center of the UV emission unit.

11. The UV sterilization device of claim 10,
wherein the lenses are disposed to be spaced apart from each other and configured such that any one of the lenses overlaps the UV emission unit by the rotation of the lens assembly.

12. The UV sterilization device of claim 9, further comprising:
a temperature sensor configured to sense a temperature in the sterilization unit; and
a controller configured to control the operation of the motor based on the temperature received from the temperature sensor.

13. The UV sterilization device of claim 12,
wherein the controller is configured to receive a sterilization mode input through an interface that communicates with the UV sterilization device, and configured to control the operation of the coin motor based on the received sterilization mode.

14. A method for controlling a UV sterilization device, the method comprising:
detecting a temperature in an enclosed space in which a sterilization unit is positioned;
withdrawing the sterilization unit from an enclosure positioned in the enclosed space when the temperature exceeds a preset threshold, by driving a motor configured to move the sterilization unit to or from the enclosure positioned in the enclosed space; and
driving the motor to return the sterilization unit to the enclosure positioned in the enclosed space when the temperature is equal to or lower than the threshold temperature.

15. The method of claim 14,
wherein the motor is driven in a first direction to withdraw the sterilization unit from the enclosure positioned in the enclosed space, and the motor is driven in a second direction, which is an opposite direction of the first direction, to return the sterilization unit into the enclosure positioned in the enclosed space.

16. The method of claim 14,
wherein the enclosed space is a vehicle, and the enclosure is an overhead console.

17. The method of claim 16, further comprising:
before the detecting of the temperature, determining whether there is a passenger in the vehicle.

18. A method for controlling a UV sterilization device, the method comprising:
receiving a sterilization request from an interface configured to communicate with the UV sterilization device of claim 1; and
driving the driving unit according to a requested sterilization mode,
wherein the sterilization unit is configured to move by a preset distance according to the requested sterilization mode.

19. The method of claim 18, further comprising: rotating the lens assembly according to the requested sterilization mode.

20. The method of claim 18,
wherein a sterilization range is adjusted by applying various lenses to the lens assembly.
